(19) United States
(12) Patent Application Publication
(10) Pub. No.: US 2023/0285308 A1
(43) Pub. Date: Sep. 14, 2023

(54) Capsule to support the body's defense against microplastics

(71) Applicants: Morgan Muchnick, Falls Church, VA (US); Timothy Chrisman, Falls Church, VA (US)

(72) Inventors: Morgan Muchnick, Falls Church, VA (US); Timothy Chrisman, Falls Church, VA (US)

(21) Appl. No.: 17/691,299

(22) Filed: Mar. 10, 2022

(51) Int. Cl. A61K 9/48; A61K 31/198; A61K 31/26; A61K 31/7048; A61K 36/752; A61K 9/20

(52) U.S. Cl. CPC A61K 9/48; A61K 31/198; A61K 31/26; A61K 31/7048; A61K 36/752; A61K 9/20

(57) ABSTRACT

This is a nutritional supplement designed to support the removal of microplastics from the stomach, intestines, liver, and kidney. It combines 5 natural compounds shown in clinical trials to bind with, and allow the body to pass microplastics more easily, into a single supplement.

### BACKGROUND OF THE INVENTION

### Field of the Invention

The present invention relates to a tablet and a method for stabilizing and removing of microplastics from the gastrointestinal and urinary systems of persons over the age of 18. The method includes administering a dosage up to two times a day of not less than 600 mg and not more than 800 mg of N-acetylcysteine, not less than 500 mg and not more than 700 mg Calcium D-Glucarate, not less than 20 mg and not more than 35 mg of Sulforaphane, not less than 200 mg and not more than 300 mg Hesperidin, not less than 400 mg and not more than 500 mg grapefruit extract (also known as citrus seed extract).

### SUMMARY OF THE INVENTION

The invention is a formulation. The ingredients are combined into a capsule. 1) 675 mg N-acetylcysteine, 2) 600 mg Calcium D-Glucarate, 3) 30 mg Sulforaphane, 4) 250 mg Hesperidin, 5) 450 mg Grapefruit Extract. The capsule is to be taken one to two times daily preferably with a meal to help the removal of microplastics from the body.

This is a nutritional supplement designed to support the removal of microplastics from the stomach, intestines, liver, and kidney. It combines 5 natural compounds shown in clinical trials to bind with, and allow the body to pass microplastics more easily, into a single supplement.

1. A tablet or capsule consisting essentially of not less than 600 mg and not more than 800 mg of N-acetylcysteine, not less than 500 mg and not more than 700 mg Calcium D-Glucarate, not less than 20 mg and not more than 35 mg of Sulforaphane, not less than 200 mg and not more than 300 mg Hesperidin, not less than 400 mg and not more than 500 mg grapefruit extract (also known as citrus seed extract).
